(19) United States
(12) Patent Application Publication
(10) Pub. No.: US 2024/0307124 A1
(43) Pub. Date: Sep. 19, 2024

(54) SYSTEM AND METHOD FOR CLAMPING GUIDANCE BASED ON GENERATED PERFUSION ZONES

(71) Applicant: Covidien LP, Mansfield, MA (US)

(72) Inventor: Faisal I. Bashir, Andover, MA (US)

(21) Appl. No.: 18/591,160

(22) Filed: Feb. 29, 2024

Related U.S. Application Data
(60) Provisional application No. 63/452,201, filed on Mar. 15, 2023.

(51) Int. Cl. A61B 34/10; A61B 1/00; A61B 34/00; A61B 34/30; A61B 90/00; G06T 7/13; G06T 7/33; G06T 15/08; G06T 17/00

(52) U.S. Cl. CPC A61B 34/10; A61B 1/000094; A61B 34/25; A61B 34/30; A61B 90/37; G06T 7/13; G06T 7/344; G06T 15/08; G06T 17/00; A61B 2034/105; A61B 2034/301; G06T 2200/04; G06T 2200/24; G06T 2207/10068; G06T 2207/20092; G06T 2207/30096; G06T 2207/30101; G06T 2207/30204; G06T 2210/41; G06T 2210/56

(57) ABSTRACT

An imaging system includes an endoscopic camera configured to acquire an intraoperative image of tissue and a blood vessel. The system also includes an image processing device coupled to the endoscopic camera. The image processing device includes a processor configured to: receive a perfusion zone model of the tissue and an operative plan including at least one clamp location; and generate an overlay of the perfusion zone model over and the at least one clamp location on the intraoperative image of the tissue and the blood vessel, respectively. The system also includes a screen configured to display the overlay and the intraoperative image.

### CROSS-REFERENCE TO RELATED APPLICATIONS

This application claims the benefit of, and priority to, U.S. Provisional Patent Application Ser. No. 63/452,201 filed on Mar. 15, 2023. The entire contents of the foregoing application are incorporated by reference herein.

### BACKGROUND

In recent years, with the advent of advanced volumetric segmentation techniques, preoperative imaging raw data from computed tomography (CT), magnetic resonance imaging (MRI), etc. have been used to generate 3D models of the surgical site. Such 3D models provide preoperative planning guidance that helps the surgeon plan the surgical approach during a surgical procedure, e.g., resect a tumor. There is an unmet need to provide surgically relevant guidance derived from the 3D model of the surgical site in both the preplanning and the intraoperative stages of the surgical procedure.

### SUMMARY

The present disclosure provides a system and method for providing surgically relevant preplanning and intraoperative guidance derived from a 3D model of a surgical site. In particular, the system and method provide preoperative guidance in the form of generative perfusion zones from 3D models as well as guidance on which blood vessels, i.e., arteries, that need to be clamped or clipped. In the pre-operative stage, the system presents the user with a user interface that also allows for modification of the automatic selective clamping location as well as the perfusion and ischemic zones. The system and method also provide intra-operative guidance to help the surgeon identify the blood vessels to be clamped while performing the surgical procedure.

According to one embodiment of the present disclosure an imaging system is disclosed. The imaging system includes an endoscopic camera configured to acquire an intraoperative image of tissue and blood vessels. The system also includes an image processing device coupled to the endoscopic camera. The image processing device includes a processor configured to: receive a perfusion zone model of the tissue and an operative plan that includes at least one clamp location; and generate an overlay of the perfusion zone model over the at least one clamp location on the intraoperative image of the tissue and the blood vessel, respectively. The system also includes a screen configured to display the overlay and the intraoperative image.

Implementations of the above embodiment may include one or more of the following features. According to one aspect of the above embodiment, the processor may be further configured to generate a depth map and a point cloud based on the intraoperative image. The processor may be further configured to register the perfusion zone model with the intraoperative image based on the depth map and the point cloud. The perfusion zone model may include an ischemic volume zone and a perfused volume zone. The processor may be further configured to register the ischemic volume zone and the perfused volume zone with an ischemic surface and a perfused surface of the tissue, respectively.

According to another embodiment of the present disclosure, a surgery planning device is disclosed. The surgery planning device includes a processor configured to receive a 3D preoperative tissue image having a 3D arterial tree, and generate a 3D perfusion model based on the 3D arterial tree. The device also includes a screen configured to display the 3D perfusion model and a graphical user interface configured to generate a selective guidance plan based on the 3D perfusion model.

Implementations of the above embodiment may include one or more of the following features. According to one aspect of the above embodiment, the processor may be further configured to verify the 3D arterial tree by generating a voxel count bounded by a vessel boundary of the 3D arterial tree. The processor may be further configured to compute a normalized vessel voxel ratio based on the voxel count. Generation of the 3D perfusion model by the processor may further include generating a skeleton model of the 3D arterial tree. Generation of the 3D perfusion model by the processor may further also include generating bifurcation points for vessels of the 3D arterial tree. Generation of the 3D perfusion model by the processor may further include computing a volumetric multi-label distance transform map based on the skeleton model. Generation of the 3D perfusion model by the processor may further include generating a tumor volume zone, an ischemic volume zone, and/or a perfused volume zone. The graphical user interface may be further be configured to display at least one virtual clamp. The graphical user interface may be further configured to update at least one parameter of the tumor volume zone, an ischemic volume zone, and/or a perfused volume zone based on a location of the at least one virtual clamp. Furthermore, the graphical user interface may allow the user to accept or modify the selective clamping location as well as the tumor volume zone, an ischemic volume zone, and/or a perfused volume zone during the pre-planning phase.

According to a further embodiment of the present disclosure, a surgical robotic system is disclosed. The system includes a robotic arm having an endoscopic camera configured to acquire an intraoperative image of tissue and a blood vessel. The system also includes an image processing device coupled to the endoscopic camera. The image processing device includes a processor configured to receive a perfusion zone model of the tissue and an operative plan having at least one clamp location. The processor is further configured to generate an overlay of the perfusion zone model over and the at least one clamp location of the intraoperative image of the tissue and the blood vessel, respectively. The system also includes a screen configured to display the overlay and the intraoperative image.

Implementations of the above embodiment may include one or more of the following features. According to one aspect of the above embodiment, the processor may be further configured to generate a depth map and a point cloud based on the intraoperative image. The processor may be further configured to register the perfusion zone model with the intraoperative image based on the depth map and the point cloud. The processor may be further configured to register the perfusion zone model with the intraoperative image based on the fusion between the kinematics data of the robotic arm and visual SLAM. The perfusion zone model may include an ischemic volume zone and a perfused volume zone. The processor may be further configured to register the ischemic volume zone and the perfused volume zone with an ischemic surface and a perfused surface of the tissue, respectively.

### BRIEF DESCRIPTION OF THE DRAWINGS

The present disclosure may be understood by reference to the accompanying drawings, when considered in conjunction with the subsequent, detailed description, in which:

FIG. 1 is a schematic diagram of a surgery planning device according to an embodiment of the present disclosure;

FIG. 2 is a 3D tissue model according to an embodiment of the present disclosure;

FIG. 3 is a flow chart of a method for generating intraoperative clamping guidance according to an embodiment of the present disclosure;

FIG. 4 is a flow chart of a method for verifying an arterial tree of the 3D tissue model for perfusion mapping according to an embodiment of the present disclosure;

FIG. 5 is a 3D artery model according to an embodiment of the present disclosure;

FIG. 6 is a processed 3D artery model according to an embodiment of the present disclosure;

FIG. 7 is a flow chart of a method for generating perfusion zones according to an embodiment of the present disclosure;

FIG. 8 is a schematic diagram an artery skeleton model used in generating perfusion zones according to an embodiment of the present disclosure;

FIG. 9 is 3D perfusion zone model according to an embodiment of the present disclosure;

FIG. 10 is a flow chart of a method for generating preoperative clamping guidance according to an embodiment of the present disclosure;

FIG. 11 is a flow chart of a method for providing intraoperative clamping guidance according to an embodiment of the present disclosure;

FIG. 12 is an image of an augmented reality overlay of the 3D perfusion zone model and clamping guidance according to an embodiment of the present disclosure;

FIG. 13 is a schematic diagram of an imaging system according to an embodiment of the present disclosure; and

FIG. 14 is a perspective view of a surgical robotic system including the imaging system according to an embodiment of the present disclosure.

### DETAILED DESCRIPTION

Embodiments of the presently disclosed system are described in detail with reference to the drawings, in which like reference numerals designate identical or corresponding elements in each of the several views. In the following description, well-known functions or constructions are not described in detail to avoid obscuring the present disclosure in unnecessary detail. Those skilled in the art will understand that the present disclosure may be adapted for use with any imaging system.

The present disclosure provides for a system and method for generating preoperative perfusion zones and surgery planning, which may then be used intraoperatively to guide clamping. Clamping during surgical procedure is commonly used in resection to cut off blood supply to a resection portion. An exemplary procedure that involves clamping is a partial nephrectomy during which a tumor is removed from a kidney. Global clamping during partial nephrectomy results in a large ischemic volume. Thus, clamping only the arteries supplying blood to the tumor would minimize the ischemic volume. The system and method generate a 3D model of vasculature from preoperative images (e.g., CT/MRI) and estimate perfusion zones based on detailed arterial trees. The system also provides planning stage guidance to clamp selective arteries and subsequently uses the perfusion zones to provide clamping guidance during the surgery to minimize ischemia.

Perfusion zones modeling enables identification of sub-arterial trees that feed different sub-volumes of organs and tumors as well as for identification of sub-volume regions fed by each sub-arterial tree. The system simulates selective clamping process and enables identification of the set of sub-arterial trees that feed the tumors and the set of sub-arterial trees that perfuse the healthy tissue. Selective clamping also allows for marking the sub-arterial trees that should be clamped to maintain healthy tissue perfusion. Selective clamping guidance may be used intraoperatively to reduce the ischemic volume, while keeping the healthy tissue perfused. The guidance may be also implemented in surgical robotic systems.

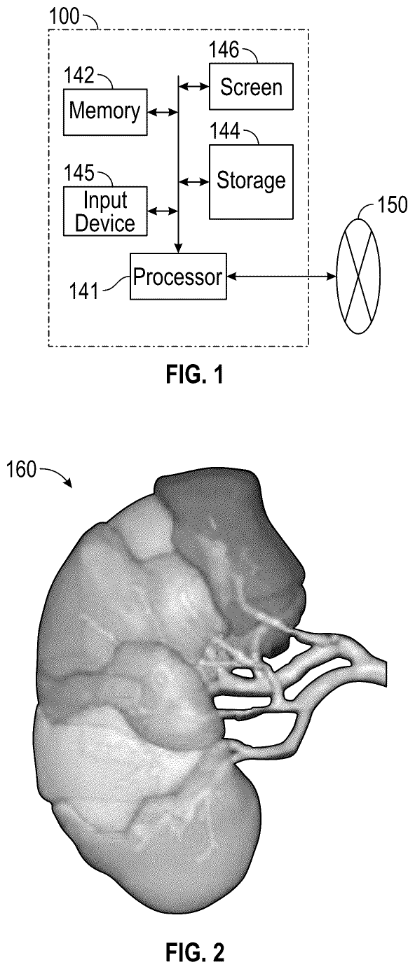

With reference to FIG. 1, a surgery planning device 100 is a computing device and can communicate with a network 150 such as a backbone LAN (local area network) in a hospital. The surgery planning device 100 includes a processor 141, a memory 142, a storage device 144, an input device 145, and a display screen 146. The processor 141 is connected to each of the hardware components constituting the surgery planning device 100.

The input device 145 may be any suitable user input device such as a keyboard, a touch screen, or a pointing device that can be operated by the operator and send input signals according to an operation to the processor 141. The processor 141 may be configured to perform operations, calculations, and/or sets of instructions described in the disclosure and may be a hardware processor, a field programmable gate array (FPGA), a digital signal processor (DSP), a central processing unit (CPU), a microprocessor, and combinations thereof. If an instruction is input by an operator such as a physician operating the input device 145, the processor 141 executes a program stored in the memory 142. The processor 141 is configured to load software instructions stored in the storage device 144 and/or transferred from the network 150 or a removable storage device (not shown) into the memory 142 to execute such instructions. The memory 142 may be a transitory storage device such as RAM (random access memory) and the like and is used as working memory for the processor 141 and used to temporarily store data.

The storage device 144 is a non-transitory storage device, e.g., hard disc drive, flash storage, etc. The storage device 144 is a storage device in which programs installed in the surgery planning device 100 (including an application program as well as an OS (operating system)) and data are stored. Also, the OS provides a GUI (graphical user interface) that displays information to the operator so that the operator can perform operations through the input device 145. The screen 146 may be any suitable monitor and may include a touchscreen that is configured to display the GUI for planning surgery.

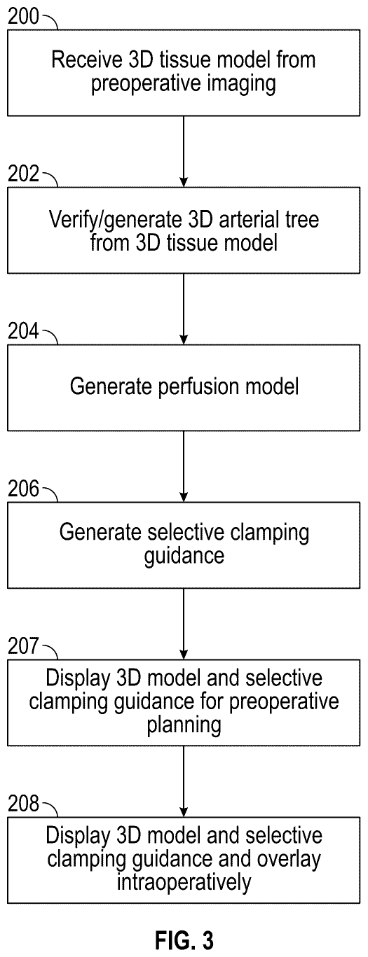

The surgery planning device 100 is configured to receive a 3D tissue or organ model 160 (FIG. 2), which is obtained using any suitable imaging modality such as computed tomography (CT), magnetic resonance imaging (MRI), or any other imaging modality capable of obtaining 3D images. With reference to FIG. 3, a flow chart shows a general method for generating perfusion zones, preoperative clamping guidance, and providing intraoperative clamping overlay. The method may be implemented as software instructions executable by the surgery planning device 100 and/or image processing unit 20 (FIG. 13). At step 200, the surgery planning device 100 receives the 3D tissue model 160, e.g., through the network 150. At step 202, the surgery planning device 100 verifies whether the 3D tissue model 160 includes a suitable 3D arterial tree.

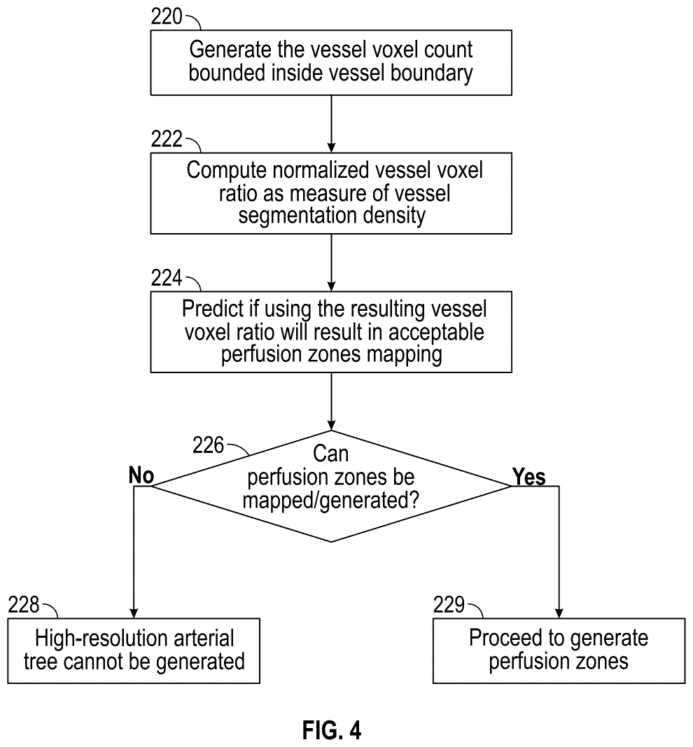

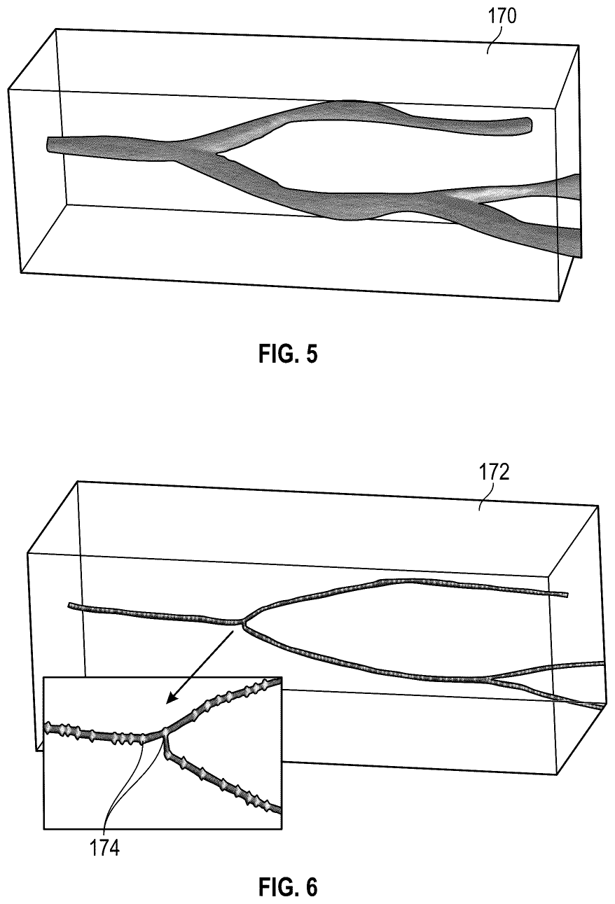

The method of FIG. 4 shows a flow chart of a method having subcomponents of step 202, which is used to verify that high-resolution arterial tree has been generated as part of 3D tissue model 160. This process takes as input the 3D tissue model 160 from pre-op imaging and generates the vessel voxel count bounded inside vessel boundary. The method next computes normalized vessel voxel ratio as a measure of vessel segmentation density. The method then predicts if using the resulting vessel voxel ratio will result in acceptable perfusion zones mapping. At step 220 the surgery planning device 100 initially generates a vessel voxel count bounded inside vessel boundary. FIG. 5 shows a 3D vessel model 170 of a vessel obtained from the 3D tissue model 160. The surgery planning device 100 generates the vessel voxel counts 174 as shown in a processed 3D vessel model 172 of FIG. 6.

At step 222, the surgery planning device 100 computes normalized vessel voxel ratio as a measure of vessel segmentation density. The surgery planning device 100 also predicts if the 3D vessel model 170 may be used to generate acceptable perfusion zones at step 224 by comparing the resulting vessel voxel ratio to a preset threshold at step 226. If the voxel ratio is below the threshold, then at step 228, the surgery planning device 100 determines that a high-resolution arterial tree cannot be generated, and thus, perfusion zones cannot be generated either. The surgery planning device 100 may then request re-creation of 3D tissue model 160 with detailed arterial tree, i.e., suitable 3D vessel model 170. If the voxel ratio is above the threshold, the surgery planning device 100 proceeds to generate perfusion zone model 190 (FIG. 9) at step 229.

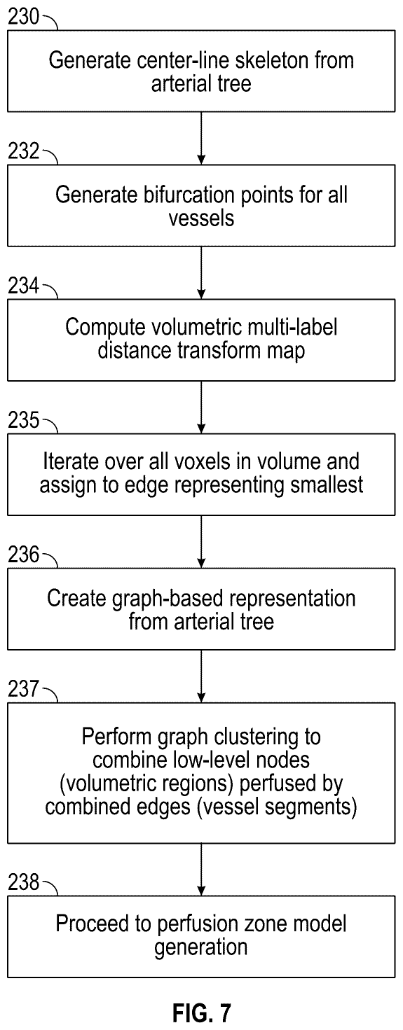

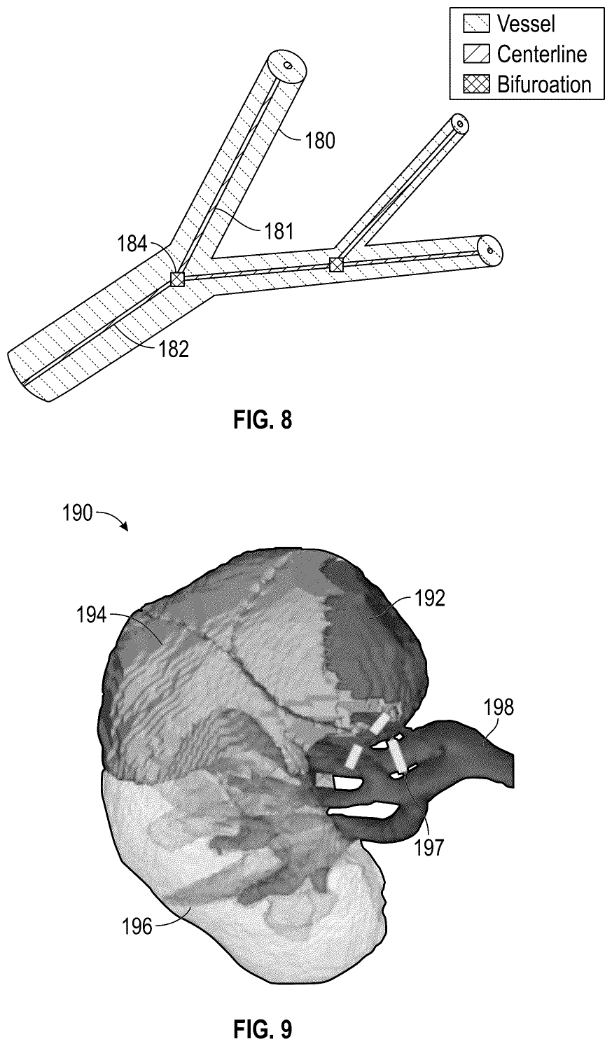

With reference to the general method of FIG. 3, at step 204 the surgery planning device 100 generates perfusion zones from the 3D vessel model 170. FIG. 7 shows a flow chart of a method having subcomponents of step 204, of generating perfusion zones from the 3D vessel model 170 with detailed arterial tree. At step 230, the surgery planning device 100 generates a center-line skeleton from arterial tree and bifurcation points at step 232 as shown in FIG. 8, in which a vessel 180 includes skeleton model 181 a centerline 182 generated through each bifurcation point 184. Each of the bifurcation points 184 is also assigned a unique label. The surgery planning device 100 also assigns a unique label to each edge (i.e., segment) disposed between bifurcation points 184. At step 234, the surgery planning device 100 further computes a volumetric multi-label distance transform map based on the skeleton model 181. At step 235, the surgery planning device 100 then iterates over all the voxels of the vessel volume. The surgery planning device 100 further creates graph-based representation from the arterial tree at step 236. In particular, the edges of the graph are centerline edges generated between bifurcation points in vessel skeleton model 181 and the nodes of the graph are volumetric sub-regions with the shortest distance to each edge.

At step 237, the surgery planning device 100 performs graph clustering to combine low-level nodes (i.e., from volumetric regions) perfused by combined edges (i.e., vessel segments). The surgery planning device 100 may use any suitable classical graph clustering algorithms or graph neural networks (GNN's) to generate perfusion zone model 190 (FIG. 9) at step 238.

With reference to the general method of FIG. 3, at step 206 the surgery planning device 100 generates selective clamping guidance and displays the perfusion zone model 190 as shown in FIG. 9. The surgery planning device 100 also provides for selective clamping guidance during preoperative planning at step 207. The perfusion zone model 190 includes a tumor volume zone 192, an ischemic volume zone 194, and a perfused volume zone 196. The blood flow is modeled in the perfusion zone model 190 based on the arterial tree and provides for simulated clamping, where virtual clamps 197 placed on arteries of an arterial tree 198 affect the simulated blood flow through the zones 192-196.

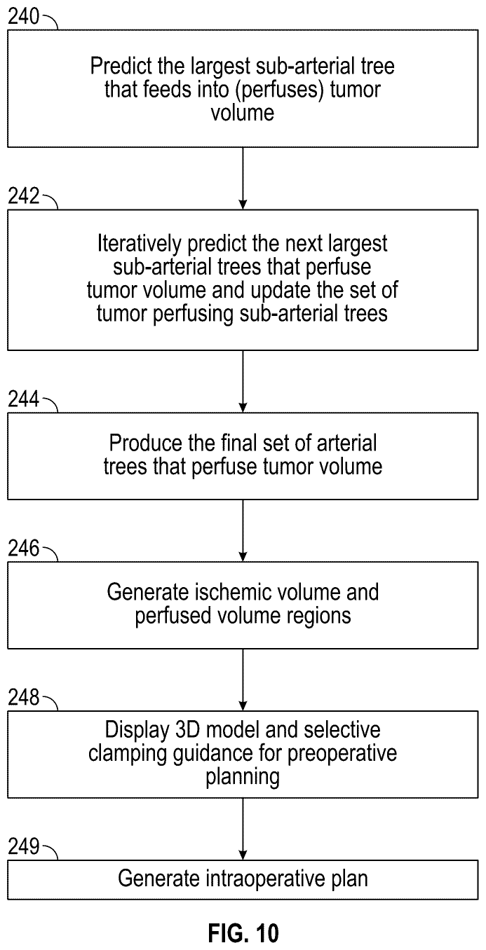

FIG. 10 shows a flow chart of a method having subcomponents of steps 206 and 207, which is used to generate clamping guidance from the perfusion zone model 190. At step 240, the surgery planning device 100 predicts the largest sub-arterial tree of the arterial tree 198 that feeds into (i.e., perfuses) tumor volume zone 192. The surgery planning device 100 also iteratively predicts the next largest sub-arterial trees that perfuse tumor volume zone 192 and updates the set of tumor perfusing sub-arterial trees at step 242. At step 244, the surgery planning device 100 produces the final set of arterial trees that perfuse tumor volume zone 192.

The surgery planning device 100 further generates the ischemic volume zone 194 and perfused volume zone 196 at step 246 and displays the perfusion zone model 190 on the screen 146 at step 248. The surgery planning device 100 may also display selective clamping guidance for preoperative planning. This may include displaying preferred locations for placing virtual clamps 197 based on the location of the tumor volume zone 192. The surgery planning device 100 may automatically identify the tumor volume zone 192 (e.g., using image processing algorithms) or the tumor volume zone 192 may be identified by the user of the surgery planning device 100 by using a GUI. The user may draw boundaries using the input device 145 around the tumor volume zone 192. The GUI and the input device 145 may be used to place, move, and/or remove the virtual clamps 197 and the surgery planning device 100 then updates the zones 192-196 based on the placement of the virtual clamps 197 in real time, i.e., the boundaries of the zones 192-196 are updated based on the placement of the virtual clamps 197. After adjusting placement of one or more virtual clamps 197 to achieve the desired size and shape of the zones 192-196, at step 249 the surgery planning device 100 generates an operative plan based on preoperative planning.

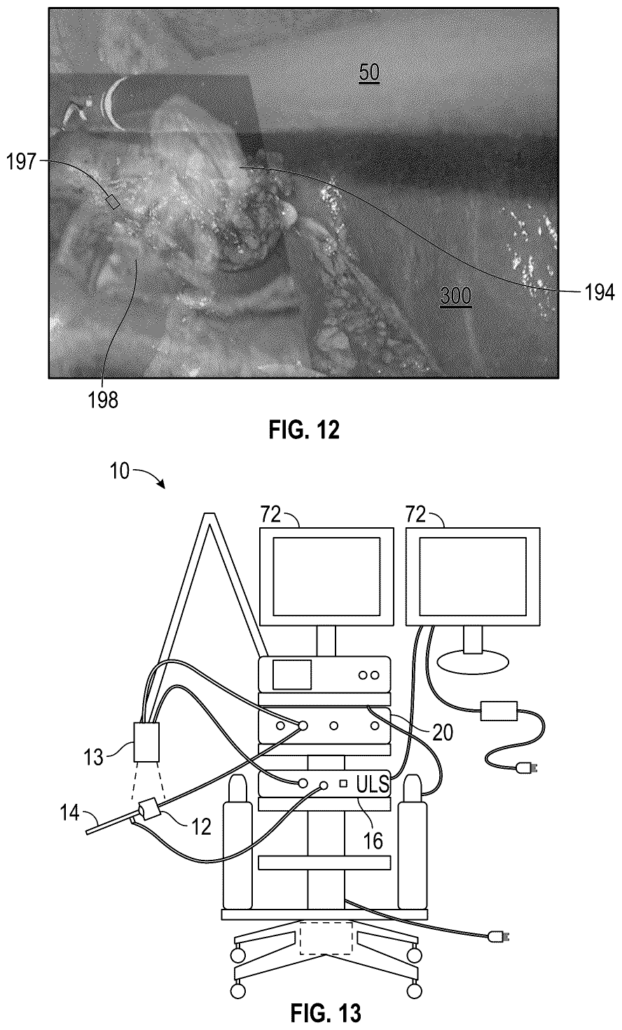

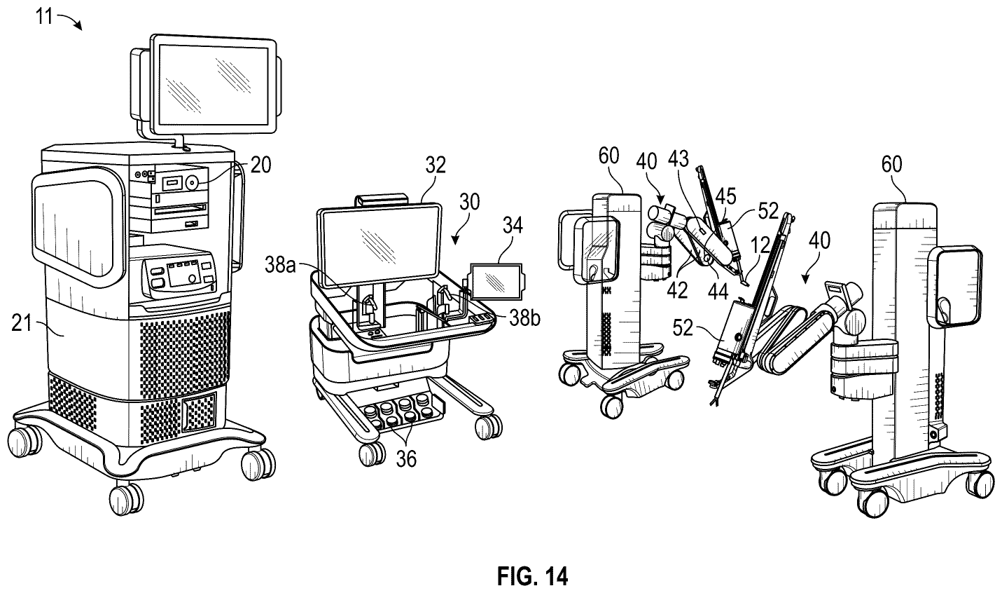

With reference to the general method of FIG. 3, at step 208 the surgery planning device 100 provides intraoperative guidance with perfusion zones and selective clamping. FIG. 12 shows a flow chart of a method having subcomponents of step 208, which is used to generate intraoperative clamping guidance based on the preoperative guidance of the perfusion zone model 190 of FIG. 9. Intraoperative guidance includes providing augmented reality overlays in real-time during the surgical procedure on a display. The augmented reality overlays may be implemented in an imaging system 10 of FIG. 13 and/or a surgical robotic system 11 of FIG. 14.

With reference to FIG. 13, the imaging system 10 includes an image processing unit 20 configured to couple to one or more cameras, such as an endoscopic camera 12 that is configured to couple to an endoscope 14 or an open surgery camera 13. The system 10 also includes a light source 16 coupled to the cameras 12 and 13. The light source 16 may include any suitable light source, e.g., white light, near infrared, etc., having light emitting diodes, lamps, lasers, etc. The endoscope 14 may be a stereoscopic endoscope.

The image processing unit 20 is configured to receive image data and process raw image data signals from the cameras 12 and 13, and generate blended white light, NIR images for recording and/or real-time display. The image processing unit 20 is also configured to blend images using various AI image augmentations.

The imaging system 10 may be also integrated with the surgical robotic system 11, which is shown in FIG. 14. A control tower 21 is connected to all of the components of the surgical robotic system 11 including a surgeon console 30 and one or more movable carts 60. Each of the movable carts 60 includes a robotic arm 40 having an attached device, which may be the endoscopic camera 12. Each of the robotic arms 40 includes a plurality of links 42 movable relative to each other about joints 44, which may have any number of degrees of freedom, e.g., one or more, providing multiple degrees of freedom to the robotic arm 40. The robotic arms 40 include actuators 45, e.g., motors, transmissions, cables, drive shafts, etc., and sensors 43 configured to provide feedback for controlling the movement of the robotic arms 40. Sensors may include electrical sensors, torque sensors, force sensors, strain sensors, temperature sensors, position sensors, and the like. Each of the robotic arms 40 also includes an instrument drive unit (IDU) 52 that is configured to couple to an actuation mechanism of the attached device and is configured to move (e.g., rotate) and actuate the device. During endoscopic procedures, the endoscopic camera 12 may be inserted through an endoscopic access port (not shown) held by the robotic arm 40.

The surgeon console 30 includes a first screen 32, which displays a video feed of the surgical site provided by camera 12, and a second screen 34, which displays a user interface for controlling the surgical robotic system 10. The first screen 32 and second screen 34 may be touchscreens (e.g., monitors 72) allowing for displaying various graphical user inputs. In embodiments, the ultrasound images may be also displayed on the first and second screens 32 and 34. The surgeon console 30 also includes a plurality of user interface devices, such as foot pedals 36 and a pair of hand controllers 38a and 38b which are used by a user to remotely control robotic arms 40 and endoscopic camera 12.

The control tower 21 also acts as an interface between the surgeon console 30 and one or more robotic arms 40. In particular, the control tower 21 is configured to control the robotic arms 40, such as to move the robotic arms 40 and the attached devices, based on a set of programmable instructions and/or input commands from the surgeon console 30, in such a way that robotic arms 40 and the attached device execute a desired movement sequence in response to input from the foot pedals 36 and the hand controllers 38a and 38b. The foot pedals 36 may be used to enable and lock the hand controllers 38a and 38b, repositioning the endoscopic camera 12. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and the attached device. This allows the user to reposition the hand controllers 38a and 38b without moving the robotic arm(s) 40 and the endoscopic camera 12. This is useful when reaching control boundaries of the surgical space.

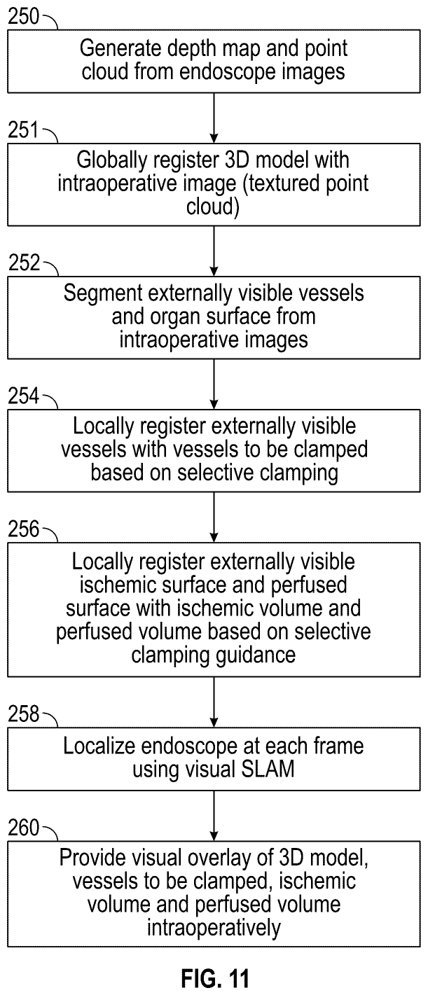

The method of FIG. 11 for generating intraoperative clamping guidance may be performed using the imaging system 10 and/or the robotic system 11. At step 250, the perfusion zone model 190 and the operative plan for selective clamping guidance is provided to the imaging system 10 and/or the robotic system 11 from the surgery planning device 100. At step 251, the image processing unit 20 generates depth map and point cloud from intraoperative endoscope images obtained by the endoscopic camera 12. Any suitable depth map generating algorithm may be used, such as depth map automatic generator (DMAG), classical approaches like Semi-Global Block Matching or Deep Learning approaches like Pyramid Stereo Matching Network (PSMNet) or Hierarchical Iterative Tile Refinement Network (HITNet) and the like for either monocular or stereo endoscopy cameras. Furthermore, at step 251, the system can use any method of global registration between the intra-operative image in the form of texture point cloud and the pre-operative 3D model. One such approach could be a semi-automatic registration of two sets of point clouds. This includes first sampling the point cloud from the 3D model to generate a point cloud representation of the 3D model, followed by automatically extracting the voxels, vertices, and meshes corresponding to the clamping location. In this semi-automatic registration approach, the system provides a user interface for the user through the robotic system 11, specifically through the hand controllers 38a and/or 38b and the first screen 32 which allows the user to point the corresponding anatomical landmarks on the endoscope video feed. In this semi-automatic registration approach, a plurality of the same anatomical points or surfaces are provided as a match between the pre-operative 3D model and the intra-operative point cloud. Finally, in step 251, any global registration approach can be used to align the pre-operative 3D model with the intra-operative textured point cloud, such as Fast Global Registration or the Iterative Closest Point (ICP) registration. Additionally, the system can train a pose estimation neural network from the pre-operative 3D model during surgery pre-planning stage and use the trained neural network to estimate the pose of the 3D model in the intra-operative scene, hence solving the global registration problem. At step 252, the image processing unit 20 globally registers the perfusion zone model 190 with intraoperative image. In embodiments, the intraoperative image and the depth map may be used to generate textured point clouds, which may be used for registration with the perfusion zone model 190. At step 254, the image processing unit 20 segments externally visible vessels and organ surfaces from intraoperative images and locally registers externally visible vessels with vessels to be clamped based on selective clamping guidance provided by the operative plan. In one embodiment, the system divides up the 3D model into multiple sub-meshes and deforms each of the sub-mesh separately in order to improve the local registration. The system can use any of the global registration approaches for the sub-meshes towards the local deformable registration.

At step 256, the image processing unit 20 also locally registers externally visible ischemic surface and perfused surface with the ischemic volume zone 194 and the perfused volume zone 196 based on selective clamping guidance provided by the operative plan. The image processing unit 20 then localizes the endoscope 14 at each frame using Visual Simultaneous Localization and Mapping (SLAM) at step 258. Visual SLAM may use robotic arm kinematics data as well as the previous and current set of images to estimate the location and pose of the endoscope 14.

At step 260, the image processing unit 20 outputs a visual, augmented reality overlay 300 over the video feed of the endoscope 14 as shown in FIG. 12. The overlay 300 includes the perfusion zone model 190 as well as vessels of the arterial tree 198 to be clamped, the tumor volume zone 192 (not shown in FIG. 12), the ischemic volume zone 194, and/or the perfused volume zone 196 (not shown in FIG. 12). The surgeon may then align a clip applier instrument 50 to place physical clamps (not shown) at the projected virtual clamps 197. The image processing unit 20 is configured to determine whether the instrument 50 is at a location corresponding to deploying the clamp at the location of the projected virtual clamps 197 and may output a prompt indicating whether the instrument 50 is at the desired location. The prompt may be a text and/or color-coded message.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. An imaging system comprising:
an endoscopic camera configured to acquire an intraoperative image of tissue and a blood vessel;
an image processing device coupled to the endoscopic camera, the image processing device including a processor configured to:
receive a perfusion zone model of the tissue and an operative plan including at least one clamp location; and
generate an overlay of the perfusion zone model over the at least one clamp location on the intraoperative image of the tissue and the blood vessel, respectively; and
a screen configured to display the overlay and the intraoperative image.

2. The imaging system according to claim 1, wherein the processor is further configured to generate a depth map and a point cloud based on the intraoperative image.

3. The imaging system according to claim 2, wherein the processor is further configured to register the perfusion zone model with the intraoperative image based on the depth map and the point cloud.

4. The imaging system according to claim 3, wherein the perfusion zone model includes an ischemic volume zone and a perfused volume zone.

5. The imaging system according to claim 4, wherein the processor is further configured to register the ischemic volume zone and the perfused volume zone with an ischemic surface and a perfused surface of the tissue, respectively.

6. The imaging system according to claim 5, wherein the processor is further configured to register the ischemic volume zone and the perfused volume zone with an ischemic surface and a perfused surface of the tissue, respectively, through a semi-automatic registration process based on landmarks identified in the intraoperative image corresponding to at least one of the at least one clamp location, the ischemic volume zone, or the perfused volume zone.

7. A surgery planning device, comprising:
a processor configured to:
receive a 3D preoperative tissue image including a 3D arterial tree; and
generate a 3D perfusion model based on the 3D arterial tree; and
a screen configured to display the 3D perfusion model and a graphical user interface configured to generate a selective clamping guidance plan based on the 3D perfusion model.

8. The surgery planning device according to claim 7, wherein the processor is further configured to receive user input to manually mark any part of the 3D arterial tree as a selective clamping location.

9. The surgery planning device according to claim 7, wherein the processor is further configured to verify the 3D arterial tree by generating a voxel count bounded by a vessel boundary of the 3D arterial tree.

10. The surgery planning device according to claim 8, wherein the processor is further configured to compute a normalized vessel voxel ratio based on a voxel count.

11. The surgery planning device according to claim 7, wherein generation of the 3D perfusion model by the processor further includes generating a skeleton model of the 3D arterial tree.

12. The surgery planning device according to claim 11, wherein generation of the 3D perfusion model by the processor further includes generating bifurcation points for vessels of the 3D arterial tree.

13. The surgery planning device according to claim 11, wherein generation of the 3D perfusion model by the processor further includes computing a volumetric multi-label distance transform map based on the skeleton model.

14. The surgery planning device according to claim 7, wherein generation of the 3D perfusion model by the processor further includes generating one or more of a tumor volume zone, an ischemic volume zone, or a perfused volume zone.

15. The surgery planning device according to claim 14, wherein the graphical user interface is further configured to display at least one virtual clamp.

16. The surgery planning device according to claim 15, wherein the graphical user interface is further configured to update at least one parameter of the tumor volume zone, an ischemic volume zone, or a perfused volume zone based on a location of the at least one virtual clamp.

17. The surgery planning device according to claim 16, wherein the graphical user interface is further configured to receive user input to least one of accept, modify, or create a new selective clamping location in the selective clamping guidance plan.

18. The surgery planning device according to claim 17, wherein the graphical user interface is further configured to receive user input to least one of accept, modify, or create a new tumor volume zone, an ischemic volume zone, or a perfused volume zone based on the tumor volume zone, the ischemic volume zone, or the perfused volume zone in the selective clamping guidance plan.

19. The surgery planning device according to claim 18, wherein the graphical user interface is further configured to output an operative plan including the 3D preoperative tissue image, selective clamping location, tumor volume zone, an ischemic volume zone, or a perfused volume zone.

20. A surgical robotic system comprising:
a robotic arm including an endoscopic camera configured to acquire an intraoperative image of tissue and a blood vessel;
an image processing device coupled to the endoscopic camera, the image processing device including a processor configured to:
receive a perfusion zone model of the tissue and an operative plan including at least one clamp location; and
generate an overlay of the perfusion zone model over the at least one clamp location over the intraoperative image of the tissue and the blood vessel, respectively; and
a screen configured to display the overlay and the intraoperative image.

21. The surgical robotic system according to claim 20, wherein the processor is further configured to generate a depth map and a point cloud based on the intraoperative image.

22. The surgical robotic system according to claim 21, wherein the processor is further configured to register the perfusion zone model with the intraoperative image based on the depth map and the point cloud.

23. The surgical robotic system according to claim 20, wherein the processor is further configured to register the perfusion zone model with the intraoperative image based on kinematics data of the robotic arm.

24. The surgical robotic system according to claim 20, wherein the perfusion zone model includes an ischemic volume zone and a perfused volume zone.

25. The surgical robotic system according to claim 24, wherein the processor is further configured to register the ischemic volume zone and the perfused volume zone with an ischemic surface and a perfused surface of the tissue, respectively.

26. The surgical robotic system according to claim 25, wherein the processor is further configured to register the ischemic volume zone and the perfused volume zone with an ischemic surface and a perfused surface of the tissue, respectively, through a semi-automatic registration process based on landmarks identified in the intraoperative image corresponding to at least one of the at least one clamp location, the ischemic volume zone, or the perfused volume zone.
